(19) United States
(12) Patent Application Publication
(10) Pub. No.: US 2015/0328221 A1
(43) Pub. Date: Nov. 19, 2015

(54) COMPOUNDS FOR PROTECTION OF PHOTORECEPTOR CELLS AND FOR ENHANCING VISUAL FUNCTION UNDER LOW LIGHT CONDITIONS

(71) Applicant: Allergan, Inc., Irvine, CA (US)

(72) Inventor: George R. Ehring, Huntington Beach, CA (US)

(21) Appl. No.: 14/710,469

(22) Filed: May 12, 2015

Related U.S. Application Data
(60) Provisional application No. 61/993,076, filed on May 14, 2014.

(51) Int. Cl. A61K 31/53; A61K 9/00; A61F 2/14; A61K 31/506

(52) U.S. Cl. CPC A61K 31/53; A61K 31/506; A61K 9/0048; A61K 9/0051; A61F 2/14

(57) ABSTRACT

Described herein are methods of enhancing visual function under low light conditions and to methods of protecting photoreceptors cells by administration of a compound selected from the group consisting of zalospirone and eptapirone; or a pharmaceutically acceptable salt thereof.

### CROSS-REFERENCE TO RELATED APPLICATIONS

This application is a non-provisional application which claims the benefit of U.S. provisional application 61/993,076 entitled “Compounds For Protection Of Photoreceptor Cells And For Enhancing Visual Function Under Low Light Conditions” filed on May 14, 2014 with docket number 19382PROV (AP) which is incorporated herein by reference in its entirety and serves as the basis for a priority and/or benefit claim of the present application.

### FIELD

The present disclosure is directed to methods of enhancing visual function under low light conditions and to methods of protecting photoreceptors cells by administration of a compound selected from the group consisting of zalospirone and eptapirone; or a pharmaceutically acceptable salt thereof.

### BACKGROUND

Early symptoms in retinal degenerations and dystrophies such as Age-related Macular Degeneration, Retinitis Pigmentosa, Nyctalopia, Cone-rod Dystrophies, Leber's Congential Amaurosis, Stargardts Disease and Vitelliform macular dystrophy includes alterations in scotopic and mesopic function indicative of a dysfunction in rod photoreceptors. In Retinitis pigmentosa, a progressive degeneration of the retina, vision gradually degenerates from the rod-rich periphery to the cone-rich center of the retina. Although in its late stages Age-related Macular Degeneration is characterized by a progressive loss of central vision, in its earliest stages patients often complain of difficulty in performing activities such as reading and driving at night or in conditions of low illumination. In this and other retinal degenerations early psychophysical and physiological manifestations in humans have demonstrated that a number of rod-mediated visual functions are compromised with age. Decreased rod-driven scotopic or mesopic vision can significantly impact the quality of life for patients with AMD and rod dystrophies.

Age-related macular degeneration (AMD) has been classified into both “dry” and “wet” (exudative, or neovascular) forms. Dry AMD is much more common than wet AMD, but the dry form can progress to the wet form, and the two occur simultaneously in a significant number of cases. Dry AMD is typically characterized by progressive apoptosis of cells in the RPE layer, overlying photoreceptor cells, and frequently also the underlying cells in the choroidal capillary layer. Confluent areas (typically at least 175 μm in minimum diameter) of RPE cell death accompanied by overlying photoreceptor atrophy are referred to as geographic atrophy. During the progression of AMD, gradual and cumulative damage to the retina occurs through various factors, including oxidative stress, RPE cell dysfunction, loss of outer photoreceptor discs, formation of intracellular (lipofuscin) and extracellular debris (drusen). Subsequent to the early reduction in photoreceptor function, loss of rod and cone cells ultimately leads to severely impaired vision or blindness in advanced cases of Age-related Macular Degeneration. The advanced form of dry AMD, or GA, is responsible for approximately 20% of all legal cases of blindness in North America with increasing incidence and prevalence owing to a higher life expectancy

Therapeutic interventions that prolong rod survival and improve their function would have a major impact on the quality of life for patients with these progressive retinal degenerative diseases, and are desirable.

EP1263504B1 U.S. Pat. No. 7,763,619 refer to the use of compounds with 5-HT1A activity useful for treating disorders of the outer retina.

### REFERENCES

Collier, R. J., Patel, Y., Martin, E. A., Dembinska, O., Hellberg, M., Krueger, D. S., Kapin, M. A., and Romano, C. Agonists at the Serotonin Receptor (5HT1A) Protect the Retina from Severe Photo-Oxidative Stress. Invest Ophthalmol. Vis. Sci. 11-18-2010.
Collier, Robert J., Wang, Yu, Smith, Sherry S., Martin, Elizabeth, Ornberg, Richard, Rhoades, Kristina, and Romano, Carmelo. Complement Deposition and Microglial Activation in the Outer Retina in Light-Induced Retinopathy: Inhibition by a 5-HT1A agonist. Investigative ophthalmology & visual science 52(11), 8108-8116. 4-5-2011.
Miyazaki, I., Asanuma, M., Murakami, S., Takeshima, M., Torigoe, N., Kitamura, Y., and Miyoshi, K. Targeting 5-HT(1A) receptors in astrocytes to protect dopaminergic neurons in Parkinsonian models. Neurobiol. Dis. 59, 244-256. 2013.
Thampi, P., Rao, H. V., Mitter, S. K., Cai, J., Mao, H., Li, H., Seo, S., Qi, X., Lewin, A. S., Romano, C., and Boulton, M. E. The 5HT(1a) Receptor Agonist 8-Oh DPAT Induces Protection from Lipofuscin Accumulation and Oxidative Stress in the Retinal Pigment Epithelium. PLoS ONE 7(4), e34468. 2012.
Marco, I., Valhondo, M., Martin-Fontecha, M., Vazquez-Villa, H., Del, Rio J., Planas, A., Sagredo, O., Ramos, J. A., Torrecillas, I. R., Pardo, L., Frechilla, D., Benhamu, B., and Lopez-Rodriguez, M. L. New serotonin 5-HT(1A) receptor agonists with neuroprotective effect against ischemic cell damage. J Med Chem. 54(23), 7986-7999. 12-8-2011.
de Freitas, R. L., Santos, I. M., de Souza, G. F., Tome, Ada R., Saldanha, G. B., and de Freitas, R. M. Oxidative stress in rat hippocampus caused by pilocarpine-induced seizures is reversed by buspirone. Brain Res Bull. 81(4-5), 505-509. 3-16-2010
Bezard, Erwan, Gerlach, Irene, Moratalla, Rosario, Gross, Christian E., and Jork, Reinhard. 5-HT1A receptor agonist-mediated protection from MPTP toxicity in mouse and macaque models of Parkinson's disease. Neurobiology of Disease 23(1), 77-86. 2006.
Berends, A. C., Luiten, P. G. M., Nyakas, C. A review of the neuroprotective properties of the 5-HT1A receptor agonist Repinotan HCl (BAY×3702 in ischemic stroke. CNS Drug Reviews 11(4) 379-402 2005.
Finger, R. P., Fenwick, E., Owsley, C., Holz, F. G., and Lamoureux, E. L. Visual functioning and quality of life under low luminance: evaluation of the German Low Luminance Questionnaire. Invest Ophthalmol. Vis. Sci. 52(11), 8241-8249. 2011.
Holz, F. G., Strauss, E. C., Schmitz-Valckenberg, S., and van Lookeren, Campagne M. Geographic Atrophy: Clinical Features and Potential Therapeutic Approaches. Ophthalmology. doi: 10.1016/j.ophtha.2013.11.023 (in press).
Berdeaux, Gilles H., Nordmann, Jean Phillipe, Colin, Emma, and Arnould, Benoit. Vision-related quality of life in patients suffering from age-related macular degeneration. American Journal of Ophthalmology 139(2), 271-279. 2005.
Hogg, R. E. and Chakravarthy, U. Visual function and dysfunction in early and late age-related maculopathy. Progress in Retinal and Eye Research 25(3), 249-276. 2006.
Schatz, H. and McDonald, H. R. Atrophic macular degeneration. Rate of spread of geographic atrophy and visual loss. Ophthalmology 96(10), 1541-1551. 1989.
Marmor, M. F., Fulton, A. B., Holder, G. E., Miyake, Y., Brigell, M., and Bach, M. ISCEV Standard for full-field clinical electroretinography (2008 update). Doc. Ophthalmol. 118(1), 69-77. 2009.

### SUMMARY

The present disclosure provides a method of enhancing visual function under low light conditions (scotopic and/or mesopic visual function) in a subject, comprising administering to the subject in need of such enhancement, a therapeutically effective amount of a compound selected from the group consisting of zalospirone and eptapirone; or a pharmaceutically acceptable salt thereof.

The present disclosure also provides a method of protecting photoreceptor cells in a subject, comprising administering to the subject in need of such protection, a therapeutically effective amount of a compound selected from the group consisting of zalospirone and eptapirone; or a pharmaceutically acceptable salt thereof.

The present disclosure also provides an ocular implant comprising a therapeutically effective amount of a compound selected from the group consisting of zalospirone and eptapirone; or a pharmaceutically acceptable salt thereof.

### BRIEF DESCRIPTION OF THE DRAWINGS

FIG. 1 shows that in the middle of the retina there is a small pit, the fovea, with which we see sharply (see: lea-test.fi/en/eyes/visfunct.html). Only a few millimeters from the fovea (arrows) the visual acuity is 20/200 (6/60 or 0.1) even in a normal person.

FIG. 2 illustrates visual field. “A” shows visual field of both eyes (see: lea-test.fi/en/eyes/visfunct.html). “B” shows that the central part of the visual field (white area) is seen by both eyes.

FIG. 3 illustrates contrast sensitivity (see: lea-test.fi/en/eyes/visfunct.html). “A” shows the contrast sensitivity curve. “B” shows visual information at different contrasts in different sizes. Note that large numbers are visible at a fainter contrast than smaller numbers.

FIGS. 4A-4D illustrate that zalospirone protects rod photoreceptors from blue light-induced injury in a model of dry AMD. Seven days after exposure to 10,000 lux blue light, the amplitude of a- and b-wave components of the electroretinogram (ERG) is reduced to 20-30% of its pre-exposure amplitude (FIG. 4A). The ERG signals from median respondents in each group are shown in the inset in A. The tracing designated “α” illustrates the signal evoked in a naïve animal when stimulated with 20 candela-sec/m² white light. The trace designated “β” shows the signal remaining 7 days after blue-light induced injury. The trace designated “γ” shows complete protection of the ERG signal in the median animal dosed with 3 mg/kg zalospirone. Full dose response showing the dose and corresponding retinal concentration of zalospirone is shown in the bar graph (FIG. 4A).

Protection of retinal architecture measured as the total count of photoreceptors (FIG. 4B) or the size of the lesion (FIG. 4C) in the section taken through the vertical meridian of the eye. (FIG. 4D) H&E stained sections of from the median respondent, naïve and vehicle- or drug-treated animals illustrate the level protection achieved by treatment with 1 mg/kg zalospirone.

FIG. 5A to 5D illustrate that eptapirone protects rod photoreceptors from blue light-induced injury in a model of dry AMD. Seven days after exposure to 10,000 lux blue light, the amplitude of a- and b-wave components of the electroretinogram (ERG) is reduced to 20-30% of its pre-exposure amplitude (A). Full dose response showing the dose and corresponding retinal concentration of eptapirone is shown in the bar graph (A). The respective doses and corresponding retinal concentrations achieved following subcutaneous administration of eptapirone are shown on the abscissa. Data is plotted as the mean±SEM percentage of a- or b-wave measured prior to blue light-induced retinal degeneration. Protection of retinal architecture measured as the area of the outer nuclear layer (B) or the size of the lesion (C) in a section taken through the vertical meridian of the eye. H&E stained sections of from the median respondent, naïve and vehicle- or drug-treated animals illustrate the level protection achieved by treatment with 0.3 mg/kg eptapirone.

FIG. 6A to 6E illustrate that zalospirone protects cone photoreceptors from blue light-induced injury in a model of dry AMD. Seven days after exposure to 10,000 lux blue light, the amplitude of the photopic b-wave, a cone-driven ERG signal, was severely reduced relative to naïve animal. The amplitude of the medium-wave (blue- and green-light responsive) cone driven ERG signals were reduced by >90% while the short-wave (ultra violet and blue-light responsive) cone signals were virtually eliminated (A, B). Pretreatment with 3 mg/kg zalospirone largely restored ERG signaling for ones (A, B). Immunofluorescent staining of short wave opsin (see hashed arrows in FIG. 6E) and medium/long wave opsin (see solid white arrows in FIG. 6C and FIG. 6E) in retinal slices illustrates that pretreatment with 3 mg/kg zalospirone also prevents the loss of cones in blue light-induced injury (FIG. 6 C-E).

FIG. 7 illustrates that zalospirone selectively enhances the scotopic b-wave. Visual function was assessed in vivo in the rat using standard ISCEV protocols. Measurements were made under scotopic (dark-adapted) conditions (“A”-“C”) or photopic conditions (“D”,“E”). In dark adapted animals, ERG recordings elicited by low intensity stimuli (1×10⁻⁴ candela-sec m²) treatment with 3 mg/kg zalospirone results in a 50% enhancement of the b-wave (“A”). However in the presence of greater intensity stimuli (20 candela-sec m²) neither the b-wave (“B”) nor the retinal oscillatory potentials (“C”) were affected by treatment with zalospirone. Under photopic conditions signals predominately driven by the cone photoreceptors, the photopic b-wave (“D”) or ERG response to 20 Hz flicker (“E”) were unaffected by treatment with zalospirone (3 mg/kg).

FIG. 8 “A” to “C” illustrates that zalospirone selectively enhances the scotopic b-wave. In dark adapted rats ERG recordings using low intensity stimuli (≦10⁻³ candelas-sec/m²) demonstrates that treatment with 3 mg/kg zalospirone preferentially augments rod-based signaling.

FIGS. 9A and 9B illustrate that treatment with zalospirone restores of the amplitude of the scotopic b-wave following light induced injury. FIG. 9A is a representative ERG recordings from a Sprague Dawley rat which was dark adapted overnight and exposed to white light stimulus (10⁻⁵ candela-sec/m²) are shown at baseline before injury, 7 days following exposure to a 3.5 hour exposure to 10,000 lux blue light and again at 3 days later following a single subcutaneous administration to 3 mg/kg zalospirone. FIG. 9B shows comparison of b-wave amplitudes at baseline, post-blue light injury, after treatment with 3 mg/kg zalospirone. The data are presented as mean±SEM, N=8. Blue light injury significantly reduced the amplitude of the b-wave from baseline (** p 0.0.01). Following injury treatment with 3 mg/kg zalospirone restored b-wave amplitudes to the baseline levels (n.s.=no significant difference).

### DETAILED DESCRIPTION

### Exemplary Embodiments

The present disclosure provides a method of enhancing visual function under low light conditions (scotopic and/or mesopic visual function) in a subject, comprising administering to the subject in need of such enhancement, a therapeutically effective amount of a compound selected from the group consisting of zalospirone and eptapirone; or a pharmaceutically acceptable salt thereof.

Zalospirone, which is a selective 5-HT_{1A} partial agonist of the azapirone chemical class, has the chemical name: (3aα,4α,4aβ,6aβ,7α,7aα)-hexahydro-2-(4-(4-(2-pyrimidinyl)-1-piperazinyl)butyl)-4,7-etheno-1H-cyclobut(f)isoindole-1,3(2H)-dione. It has the following chemical structure:

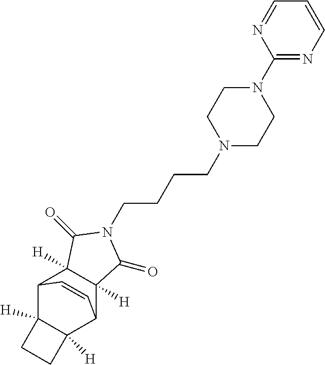 [SMILES: O=C1[C@@H]2C3C=CC([C@H]4CC[C@@H]34)[C@@H]2C(=O)N1CCCCN1CCN(c2ncccn2)CC1]

The synthesis and characterization of zalospirone and related compounds and their antagonist activity as ligands for the 5-HT_{1A} receptor are disclosed in U.S. Pat. No. 4,892,943.

Eptapirone, which is a very potent and highly selective 5-HT_{1A} receptor full agonist of the azapirone class, has the chemical name: 4-Methyl-2-[4-(4-pyrimidin-2-yl-piperazin-1-yl)-butyl]-2H-[1,2,4]triazine-3,5-dione. It has the following chemical structure:

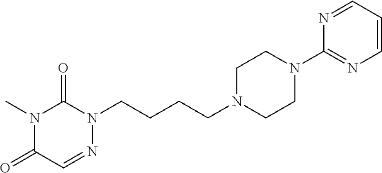 [SMILES: Cn1c(=O)cnn(CCCCN2CCN(c3ncccn3)CC2)c1=O]

The synthesis and characterization of eptapirone and related compounds and their affinity towards the 5-HT_{1A} receptor and their pharmacological profile are disclosed in U.S. Pat. No. 5,977,106.

The term “salt(s)”, as employed herein, includes acid addition salts formed with inorganic and/or organic acids. Pharmaceutically acceptable (i.e., non-toxic, physiologically acceptable) salts are preferred, although other salts are also useful. A salt of the present compound may be formed, for example, by reaction of the free base of the present compound with an amount of acid, such as an equivalent amount, in a medium such as one in which the salt precipitates, or in an aqueous medium followed by lyophilization. Suitable acid addition salts include those of the acid selected from succinic acid, hydrobromic acid, acetic acid, fumaric acid, maleic acid, methane sulfonic acid, lactic acid, phosphoric acid, hydrochloric acid, sulfuric acid, tartaric acid and citric acid. Mixtures of the acid addition salts may also be used.

Additionally, acids which are generally considered suitable for the formation of pharmaceutically useful salts from basic pharmaceutical compounds are discussed, for example, by P. Stahl et al, Camille G. (eds.) Handbook of Pharmaceutical Salts. Properties, Selection and Use. (2002) Zurich: Wiley-VCH; S. Berge et al, Journal of Pharmaceutical Sciences (1977) 66(1) 1-19; P. Gould, International J. of Pharmaceutics (1986) 33 201-217; Anderson et al, The Practice of Medicinal Chemistry (1996), Academic Press, New York; and in The Orange Book (Food & Drug Administration, Washington, D.C. on their website). These disclosures are incorporated herein by reference thereto.

All such acid salts are intended to be pharmaceutically acceptable salts within the scope of the disclosure and are considered equivalent to the free forms of the corresponding compounds for purposes of the disclosure.

Vision is composed of many simultaneous functions. If vision is normal, seeing is so effortless that we do not notice the different visual functions.

The different components of the visual image are: forms, colors and movement. Thus we have form perception, color perception and motion perception.

We see both during the day light and during very dim light. In day light, photopic vision, we perceive colors because of function of the cone cells; in very dim light, scotopic vision, we see only shades of gray, since rod cells respond only to luminance differences. In twilight, when both rod and cone cells function, we have mesopic vision.

Vision is measured with many different tests, such as tests for visual acuity, visual field, contrast sensitivity, color vision, visual adaptation to different luminance levels, binocular vision and stereoscopic vision.

The term “visual function” as used herein includes all of the above, namely visual acuity, visual field, contrast sensitivity, color vision, visual adaptation to different luminance levels, binocular vision and three dimensional (stereoscopic) vision.

In another embodiment, the visual function is visual acuity.

Information on different visual functions is available on the web at lea-test.fi/en/eyes/visfunct.html.

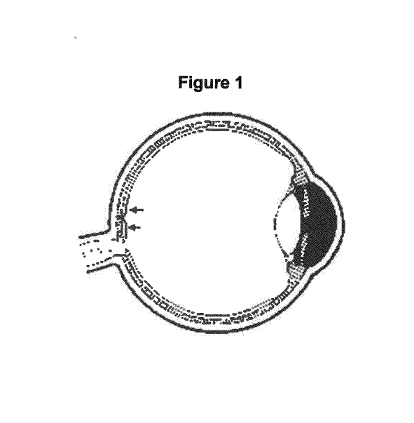

“Visual acuity” is measured with visual acuity charts at distance and at near. The test measures what is the smallest letter, number or picture size that the patient still sees correctly. Visual acuity is good only in the very middle of the retina. See FIG. 1.

When a person with normal vision looks straight forward without moving the eyes, (s)he sees also on both sides. The area visible at once, without moving the eyes, is called “visual field”. Nerve fibers from both eyes are divided so that fibers from the right half of both eyes reach the right half of the brain and fibers from the left half of both eyes the left half of the brain.

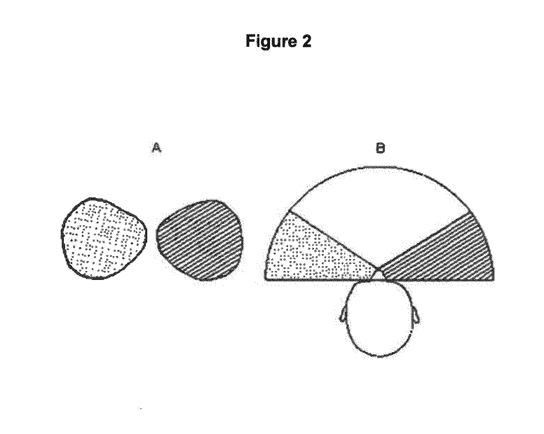

Visual information coming from both eyes is fused in the visual cortex in the back of the brain. The central part of the visual field is seen by both eyes (FIG. 2). On both sides of this central, binocular field there are half-moon formed parts of visual field that are seen by only one eye. See FIG. 2.

We use our peripheral or side vision when moving around. The most central part of the visual field is used in sustained near work, e.g., reading. When the visual field is measured with the clinical instruments these instruments measure what the weakest light is that the eye still can see in different parts of the visual field. A measurement like this gives valuable information on diseases of the visual pathways related to glaucoma or neurologic diseases. It does not give information on how the person sees forms or perceives movement in the different parts of the visual field.

The visual field can change in many ways. Therefore it is often difficult to understand how a visually impaired person sees. If the side parts of the visual field function poorly the person may need to use a white cane in order to move around safely, but (s)he may be able to read without glasses. On the other hand, if the side parts function well and the central field functions poorly, the person may walk like a normally sighted person, but may be able to read only the headings of a newspaper.

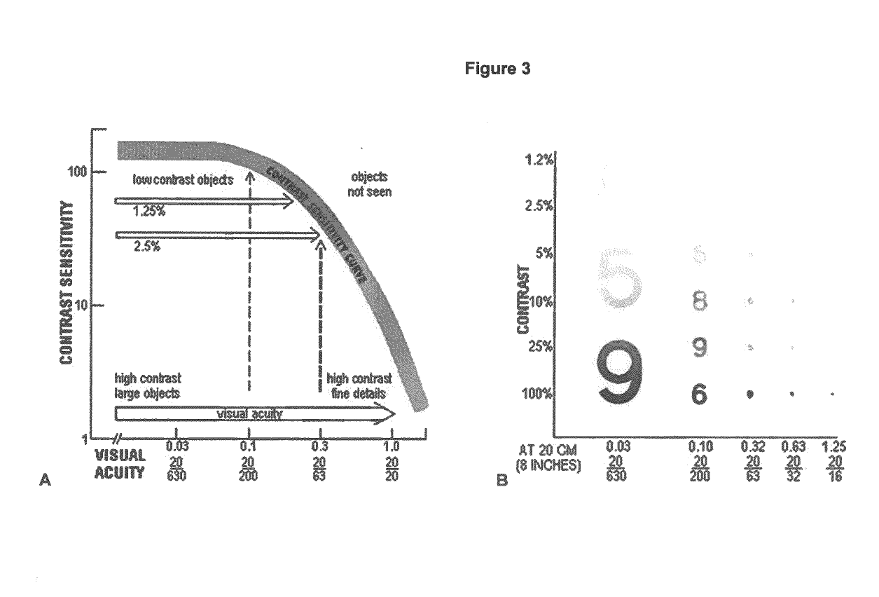

“Contrast sensitivity” can be depicted, for example, by a curve (See FIG. 3A). Under the curve there are the objects that we can see, above and to the right of the slope of the curve is the visual information that we cannot see. Contrast sensitivity can be measured using striped patterns, gratings, or symbols at different contrast levels.

When we measure hearing, an audiogram depicts which are the weakest tones at different frequencies that we still can hear. The measurements are made at low, intermediate and high frequencies. When we measure contrast sensitivity we measure what is the faintest grating or symbol still visible when the symbols are large, medium size or small (FIG. 3B).

If a visually impaired person has poor contrast sensitivity (s)he cannot see small contrast differences between adjacent surfaces. Everything becomes flat. It is difficult to perceive facial features and expressions. Text in the newspapers seems to have less contrast than before and it is difficult to recognize the edge of the pavement and the stairs.

Contrast sensitivity decreases in several common diseases, diabetes, glaucoma, cataract and diseases of the optic nerve.

Visual Adaptation to Different Luminance Levels:

A normally sighted person can read by one candle's light and (s)he can read in bright sun light. The difference in the amount of light present in these two situations is million times. The normal person can adapt his/her vision to function at the different luminance levels.

The rod cells of the retina see best in twilight. If they do not function, the person is night blind. Night blindness is the first symptom that develops in many retinal diseases. First the child with a retinal disease starts to see in dim light after an abnormally long waiting. Therefore (s)he will have difficulties in finding his/her clothing in a closet or in a drawer if there is no extra illumination directed into these places. Later (s)he loses night vision completely, even when waiting for a long time (s)he does not start to see in the dark. Changes in visual adaptation time can be easily detected with a dark adaption test, which measures the rate at rod and cones recover sensitivity in the dark after exposure to a bright light source.

Photophobia and delayed adaptation to bright light are often additional symptoms of abnormal visual adaptation. When normally sighted persons enter from a darker room into a bright light, they also see very little for a second, sometimes it even hurts their eyes. They are dazzled. A visually impaired person may be dazzled for a long time. It is possible to decrease the problem by using absorptive glasses and a hat with wide brim or a visor.

Variation in the Nature of Visual Disability:

Different visual functions may become impaired independent of each other. Therefore there are many different types of visual impairment and disability. Sometimes a visually impaired person seems to function in a very confusing way. One moment (s)he seems to function like a normally sighted person and in the next moment like a blind person. A visually impaired person seldom pretends to see less than what (s)he actually sees.

One reason for variation in visual behavior might be changes in illumination. Another may be that (s)he knows the surroundings so there is no difficulty in orientation. Normally sighted persons move about the same way at home in the dark. They move confidently and securely as long there is nothing unexpected in their way. If somebody leaves an object on the usual path they may trip over it. In the very same way a visually impaired person needs only a few visual cues in a well-known place in order to be able to move freely.

If it is difficult to understand how a visually impaired person sees it is quite proper to ask him/her about his/her vision. Most visually impaired people are able to describe the nature of their impairment so well that it is possible to understand their situation better. Some persons say that they have only 10% vision left. Such a number does not describe the degree of visual impairment. The person may be able to move freely relying on his/her vision or may function like a nearly blind. That number (10%) usually means that his/her visual acuity is 20/200 (6/60 or 0.1) and it describes only one of many visual functions.

In another embodiment, the visual function is selected from the group consisting of visual acuity, visual field, contrast sensitivity, and visual adaptation to different luminance levels.

In another embodiment, the visual function is visual acuity or contrast sensitivity.

In another embodiment, the subject in need of said enhancement of visual function is one who has low/poor visual function resulting from a retinal degeneration or dystrophy.

In another embodiment, the retinal degeneration or dystrophy is selected from the group consisting of age related macular degeneration (AMD) (including wet and dry AMD), geographic atrophy, retinitis pigmentosa, nyctalopia, cone-rod dystrophy, congenital stationary night blindness, macula edema, Stargardt's disease cone dystrophy, and, macular edema, retinal detachment and tears, retinal trauma, retinal tumors and retinal diseases associated with said tumors, and diabetic retinopathy.

In another embodiment, the retinal degeneration or dystrophy is selected from the group consisting of age related macular degeneration (AMD) (including wet and dry AMD), geographic atrophy, retinitis pigmentosa, nyctalopia, and cone-rod dystrophy.

The present disclosure also provides a method of protecting photoreceptor cells in a subject, comprising administering to the subject in need of such protection, a therapeutically effective amount of a compound selected from the group consisting of zalospirone and eptapirone; or a pharmaceutically acceptable salt thereof.

In one embodiment, the photoreceptor cells are rods and cones.

In another embodiment, the subject in need of protection of photoreceptor cells is one who is suffering from a retinal degeneration or dystrophy.

In another embodiment, the retinal degeneration or dystrophy is selected from the group consisting of age related macular degeneration (AMD) (including wet and dry AMD), geographic atrophy, retinitis pigmentosa, nyctalopia, cone-rod dystrophy, macula edema, Stargardt's disease cone dystrophy, macular edema, retinal detachment and tears, retinal trauma, retinal tumors and retinal diseases associated with said tumors, diabetic retinopathy and optic neuropathies.

In another embodiment, the retinal degeneration or dystrophy is selected from the group consisting of age related macular degeneration (AMD) (including wet and dry AMD), geographic atrophy, retinitis pigmentosa, nyctalopia, and cone-rod dystrophy.

In another embodiment, the administration of the compound of the present disclosure maintains outer nuclear cell count and/or normalizes retinal structure in the face of a severe insult.

The compounds described herein can be administered in any one of conventional modes of pharmaceutical delivery, such as oral, intravenous, sublingual, intravitreal, topical, subcutaneous, trans-dermal, buccal, and intrathecal, or suitable combinations thereof. The topical and transdermal compositions can take the form of creams, lotions, aerosols and/or emulsions and can be included in a transdermal patch of the matrix or reservoir type as are conventional in the art for this purpose.

For preparing pharmaceutical compositions from the compounds described herein, inert, pharmaceutically acceptable carriers can be either solid or liquid. Solid form preparations include powders, tablets, dispersible granules, capsules, cachets and suppositories. The powders and tablets may be comprised of from about 5 to about 95 percent active ingredient. Suitable solid carriers are known in the art, e.g., magnesium carbonate, magnesium stearate, talc, sugar or lactose. Tablets, powders, cachets and capsules can be used as solid dosage forms suitable for oral administration. Examples of pharmaceutically acceptable carriers and methods of manufacture for various compositions may be found in A. Gennaro (ed.), Remington's Pharmaceutical Sciences, 18^{th} Edition, (1990), Mack Publishing Co., Easton, Pa.

Liquid form preparations include solutions, suspensions and emulsions. As an example may be mentioned water or water-propylene glycol solutions for parenteral injection or addition of sweeteners and opacifiers for oral solutions, suspensions and emulsions. Liquid form preparations may also include solutions for intranasal administration.

Aerosol preparations suitable for inhalation may include solutions and solids in powder form, which may be in combination with a pharmaceutically acceptable carrier, such as an inert compressed gas, e.g. nitrogen.

Also included are solid form preparations that are intended to be converted, shortly before use, to liquid form preparations for either oral or parenteral administration. Such liquid forms include solutions, suspensions and emulsions.

In addition to the common dosage forms set out above, the compounds described herein may also be administered by controlled release means and/or delivery devices such as those described in U.S. Pat. Nos. 3,845,770; 3,916,899; 3,536,809; 3,598,123; 3,630,200; 4,008,719; and 5,366,738 the disclosures of which are incorporated herein by reference.

For use where a composition for intravenous administration is employed, a suitable daily dosage range for anti-inflammatory, anti-atherosclerotic or anti-allergic use is from about 0.001 mg to about 25 mg (preferably from 0.01 mg to about 1 mg) of a compound of described herein per kg of body weight per day and for cytoprotective use from about 0.1 mg to about 100 mg (preferably from about 1 mg to about 100 mg and more preferably from about 1 mg to about 10 mg) of a compound described herein per kg of body weight per day. For the treatment of diseases of the eye, ophthalmic preparations for ocular administration comprising 0.001-1% by weight solutions or suspensions of the compounds described herein in an acceptable ophthalmic formulation may be used.

Preferably, the pharmaceutical preparation is in a unit dosage form. In such form, the preparation is subdivided into suitably sized unit doses containing appropriate quantities of the active component, e.g., an effective amount to achieve the desired purpose.

The magnitude of prophylactic or therapeutic dose of a compound described herein will, of course, vary with the nature of the severity of the condition to be treated and with the particular compound and its route of administration. It will also vary according to the age, weight and response of the individual patient. It is understood that a specific daily dosage amount can simultaneously be both a therapeutically effective amount, e.g., for treatment to slow progression of an existing condition, and a prophylactically effective amount, e.g., for prevention of condition.

The quantity of active compound in a unit dose of preparation may be varied or adjusted from about 0.001 mg to about 500 mg. In one embodiment, the quantity of active compound in a unit dose of preparation is from about 0.01 mg to about 250 mg. In another embodiment, the quantity of active compound in a unit dose of preparation is from about 0.1 mg to about 100 mg. In another embodiment, the quantity of active compound in a unit dose of preparation is from about 1.0 mg to about 100 mg. In another embodiment, the quantity of active compound in a unit dose of preparation is from about 1.0 mg to about 50 mg. In still another embodiment, the quantity of active compound in a unit dose of preparation is from about 1.0 mg to about 25 mg.

The actual dosage employed may be varied depending upon the requirements of the patient and the severity of the condition being treated. Determination of the proper dosage regimen for a particular situation is within the skill of the art. For convenience, the total daily dosage may be divided and administered in portions during the day as required.

The amount and frequency of administration of the compounds described herein and/or the pharmaceutically acceptable salts thereof will be regulated according to the judgment of the attending clinician considering such factors as age, condition and size of the patient as well as severity of the symptoms being treated. A typical recommended daily dosage regimen for oral administration can range from about 0.01 mg/day to about 2000 mg/day of the compounds described herein. In one embodiment, a daily dosage regimen for oral administration is from about 1 mg/day to 1000 mg/day. In another embodiment, a daily dosage regimen for oral administration is from about 1 mg/day to 500 mg/day. In another embodiment, a daily dosage regimen for oral administration is from about 100 mg/day to 500 mg/day. In another embodiment, a daily dosage regimen for oral administration is from about 1 mg/day to 250 mg/day. In another embodiment, a daily dosage regimen for oral administration is from about 100 mg/day to 250 mg/day. In still another embodiment, a daily dosage regimen for oral administration is from about 1 mg/day to 100 mg/day. In still another embodiment, a daily dosage regimen for oral administration is from about 50 mg/day to 100 mg/day. In a further embodiment, a daily dosage regimen for oral administration is from about 1 mg/day to 50 mg/day. In another embodiment, a daily dosage regimen for oral administration is from about 25 mg/day to 50 mg/day. In a further embodiment, a daily dosage regimen for oral administration is from about 1 mg/day to 25 mg/day. The daily dosage may be administered in a single dosage or can be divided into from two to four divided doses.

In a some embodiment, the compound described herein is administered intraviteally (e.g., by injection into the back of the eye).

For intravitreal administration, the weight of the device (i.e., drug plus carrier/vehicle/excipient) is typically 1 mg (which for example may be administered with a 22 G needle) and the drug load is normally 10-50%. The drug dose range for intravitreal administration is normally about 100-500 μg. However, the drug load can be stretched to 2-65%, i.e., a drug dose range of 20-650 μg can be used. However, the device weight may be 1.5 mg, and for this a drug dose range of 20-975 μg can be used.

Another way of intravitreal delivery is by injecting drug suspension formulation. For this, the dose range is 10-600 ug.

The present disclosure also provides an ocular (intraocular) implant comprising a therapeutically effective amount of a compound selected from the group consisting of zalospirone and eptapirone; or a pharmaceutically acceptable salt thereof.

The ocular implant of the present disclosure typically comprises a therapeutically effective amount of the presently disclosed compounds (the therapeutic component; the active pharmaceutical ingredient (API)), and a drug release sustaining polymer component associated with the therapeutic compound. As used herein, an “intraocular implant” refers to a device or element that is structured, sized, or otherwise configured to be place in an eye. Intraocular implants are generally biocompatible with physiological conditions of an eye and do not cause adverse side effects. Intraocular implants may be place in an eye without disrupting vision of the eye.

The implant may be solid, semisolid, or viscoelastic. The drug release sustaining component is associated with the therapeutic component to sustain release of an amount of the therapeutic component into an eye in which the implant is placed.

The therapeutic component may be released from the implant by diffusion, erosion, dissolution or osmosis. The drug release sustaining component may comprise one or more biodegradable polymers or one or more non-biodegradable polymers. Examples of biodegradable polymers of the present implants may include poly-lactide-co-glycolide (PLGA and PLA), polyesters, poly (ortho ester), poly(phosphazine), poly(phosphate ester), polycaprolactone, natural polymers such as gelatin or collagen, or polymeric blends. The amount of the therapeutic component is released into the eye for a period of time greater than about one week after the implant is placed in the eye and is effective in reducing or treating an ocular condition.

In one embodiment, the intraocular implant comprises a therapeutic component and a biodegradable polymer matrix. The therapeutic component is associated with a biodegradable polymer matrix that degrades at a rate effective to sustain release of an amount of the therapeutic component from the implant effective to treat an ocular condition. The intraocular implant is biodegradable or bioerodible and provides a sustained release of the therapeutic component in an eye for extended periods of time, such as for more than one week, for example for about one month or more and up to 5 about six months or more. The implant may be configured to provide release of the therapeutic component in substantially one direction, or the implant may provide release of the therapeutic component from all surfaces of the implant.

The biodegradable polymer matrix of the foregoing implant may be a mixture of biodegradable polymers or the matrix may comprise a single type of biodegradable polymer. For example, the matrix may comprise a polymer selected from the group consisting of polylactides, poly(lactide-co-glycolides), polycaprolactones, and combinations thereof.

In another embodiment, the intraocular implant comprises the therapeutic component and a polymeric outer layer covering the therapeutic component. The polymeric outer layer includes one or more orifices or openings or holes that are effective to allow a liquid to pass into the implant, and to allow the therapeutic component to pass out of the implant.

The therapeutic component is provided in a core or interior portion of the implant, and the polymeric outer layer covers or coats the core. The polymeric outer layer may include one or more non-biodegradable portions. The implant can provide an extended release of the therapeutic component for more than about two months, and for more than about one year, and even for more than about five or about ten years. One example of such a polymeric outer layer covering is disclosed in U.S. Pat. No. 6,331,313.

In one embodiment, the present implant provides a sustained or controlled delivery of the therapeutic component at a maintained level despite the rapid elimination of the therapeutic component from the eye. For example, the present implant is capable of delivering therapeutically effective amounts of the therapeutic component for a period of at least about 30 days to about a year despite the short intraocular half-lives that may be associated with the therapeutic component. Plasma levels of the therapeutic component obtained after implantation may be extremely low, thereby reducing issues or risks of systemic toxicity. The controlled delivery of the therapeutic component from the present implants would permit the therapeutic component to be administered into an eye with reduced toxicity or deterioration of the blood-aqueous and blood-retinal barriers, which may be associated with intraocular injection of liquid formulations containing the therapeutic component.

A method of making the present implant involves combining or mixing the therapeutic component with a biodegradable polymer or polymers. The mixture may then be extruded or compressed to form a single composition. The single composition may then be processed to form individual implants suitable for placement in an eye of a patient.

Another method of making the present implant involves providing a polymeric coating around a core portion containing the therapeutic component, wherein the polymeric coating has one or more holes. The implant may be placed in an ocular region to treat a variety of ocular conditions, such as treating the conditions disclosed herein.

The daily dose may be administered as single dose or in divided doses and, in addition, the upper limit can also be exceeded when this is found to be indicated.

### Examples

### Assays

### Electroretinogram (ERG)

Bilateral flash ERG recordings on the dual dome Espion E³ system (Diagnosys, LLC) were made prior to (baseline) and on the Day of zalospirone administration. Animals were anesthetized with a cocktail of 30 mg/mL Ketamine/0.2 mg/mL Dexmedetomidine (i.m.) to reach a final dose of 15 mg/kg Ketamine and 0.1 mg/kg Dexmedetomidine. Animals' eyes were given 1 drop each of 1% phenylephrine, 0.5% atropine and 1% proparacaine, sequentially. Noninvasive platinum wire loop electrodes moisturized with artificial tears (Goniovisc, Sigmapharmaceuticals, Inc.) were placed over the eyes, a reference electrode were placed in the bridge of the nose and a ground wire into the tail. Scotopic ERG measurements were made at 1e⁻⁵ to 20 cd s/m². Photopic ERG measurements for S- and M-cone function were made at 8e⁻⁴ to 40 cd s/m². The recording filter was set at 300 Hz.

### Tissue Processing

On Day 7 post BL-exposure, animals were anesthetized with Euthasol and decapitated. Following decapitation, bilateral globes were enucleated and marked at the 12:00 position with black tissue ink. Both eyes from each animal were incubated in 5 mL of Davidson's fixative special overnight at RT. After ˜24 hours, both eyes were dehydrated in 50% ethanol (EtOH) for 1 hour at RT followed by storage in 70% EtOH at RT until paraffin processing. Eyes were then shipped to Premier Lab (Longmont, Colo.) for paraffin processing and embedding. Five micrometer thick paraffin sections were cut through the optic nerve head and mounted on glass.

### Histological Analysis (H+E)

For each eye, three H&E-stained slides were generated. Slides containing H&E-stained cross sections were imaged using the Nanozoomer whole slide scanner employing a 40× objective under bright field optics. ONL thickness, photoreceptor column height, ONL area per tissue section, and total photoreceptor number per tissue section was quantified across the inferior to superior axis of each retinal section in digital whole slide images using the EyeAnalyzer algorithm.

### Anti-Opsin Immunofluorescence in Retinal Cross Sections

A subset of 5 μm thick paraffin sections cut through the vertical meridian (prepared by Premier Labs) was not stained with H&E, but instead was subjected to anti-opsin immunofluorescent staining to detect both SW and M/L cones. Briefly, sections were baked at 60° C. for 1 hr, deparaffinized in xylene, rehydrated in a decreasing series of alcohols, rinsed in PBS 3×, and blocked in PBS containing 0.1% Tween 20, 0.1% Triton×100, and 5% normal donkey serum for 2 hr at RT. Slides were reacted 0/N at 4° C. with a combined mixture of anti-SW (SCBT; SC-14363) and -M/L (Millipore, Ab5405) opsin antibodies diluted to 1:200 and 1:500, respectively in a PBS buffer containing 0.1% Tween 20, 0.1% Triton×100, and 5% normal donkey serum. To control for specificity of staining, an additional subset of slides were reacted with a combined mixture of anti-SW opsin antibody preadsorbed with sc-14363/blocking peptide and normal rabbit IgG. Slides were rinsed with PBS 3× and reacted with AlexaFluor 568 donkey anti-goat and AlexaFluor 488 donkey anti-rabbit secondary antibodies (diluted 1:200 in primary antibody diluent) for 1 hr at RT. Following three rinses in PBS, slides were coverslipped using Prolong mounting medium containing DAPI, dried O/N in the dark and sealed with nail polish. For each eye, two slides were stained to detect cones as detailed above. The number of SW and M/L cones was manually counted for both slides from each study eye in dual channel fluorescent images.

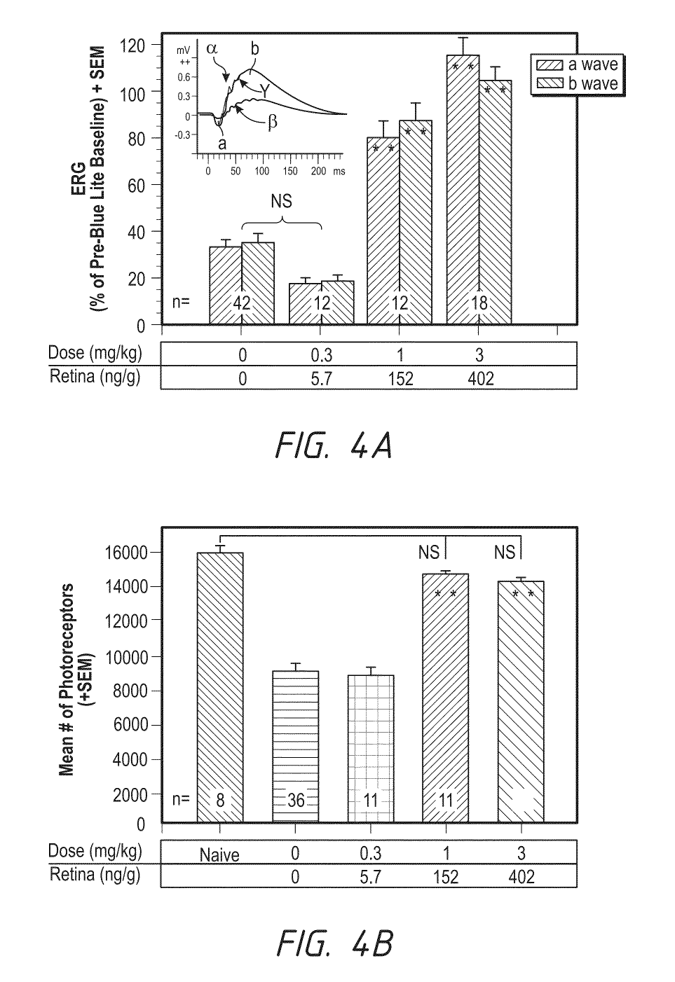

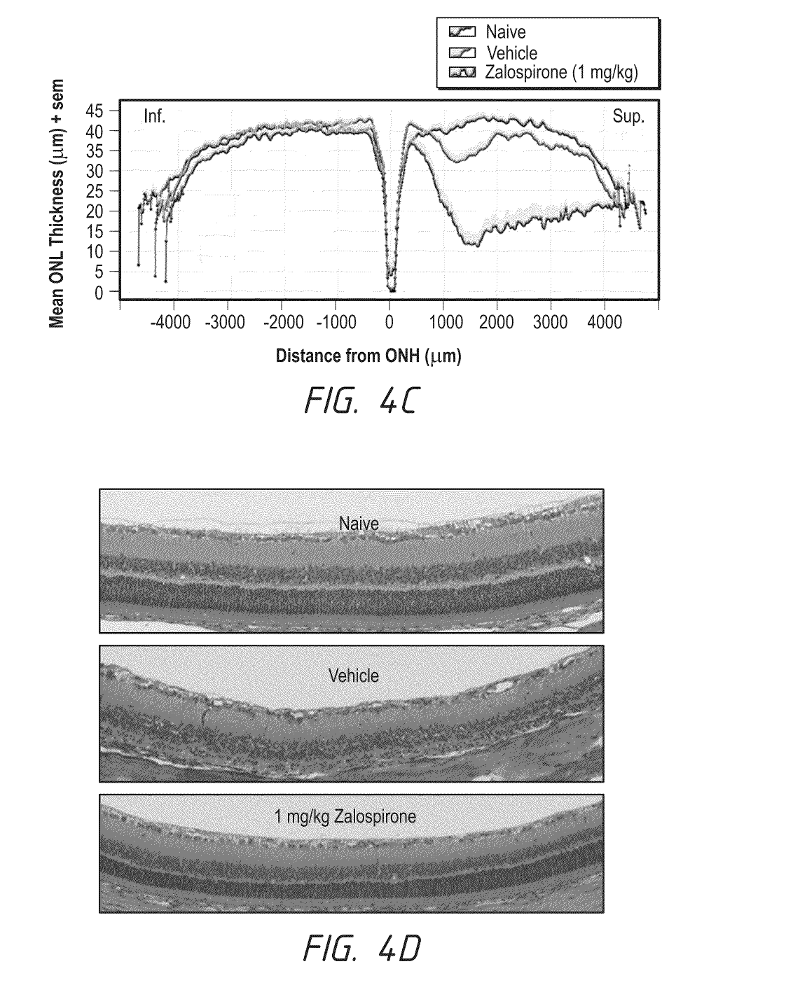

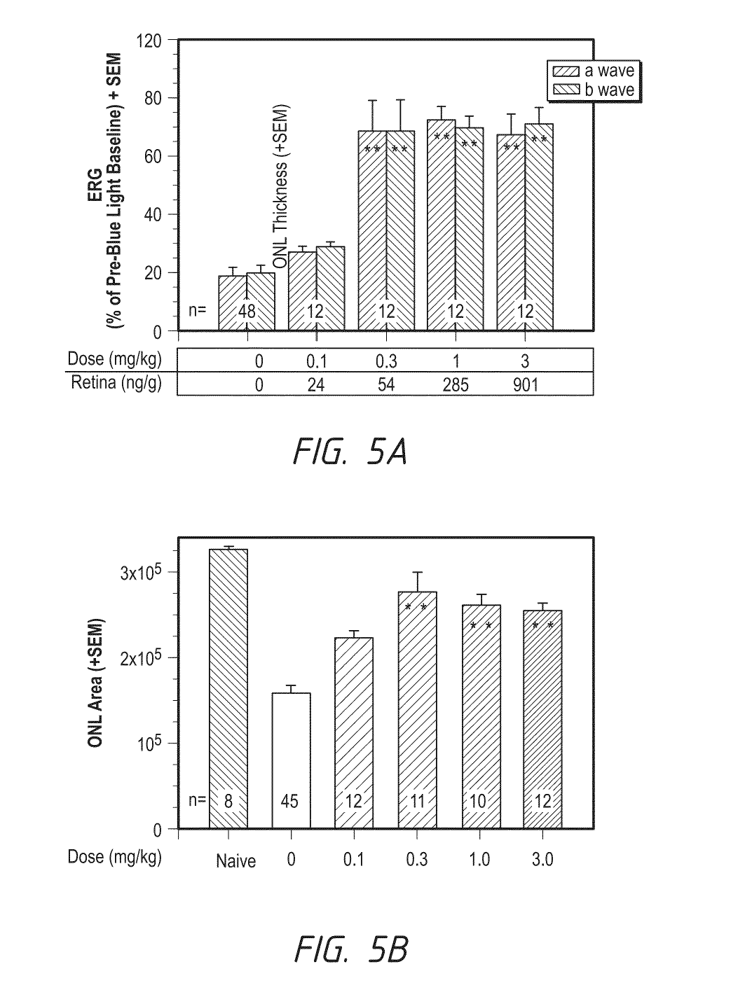

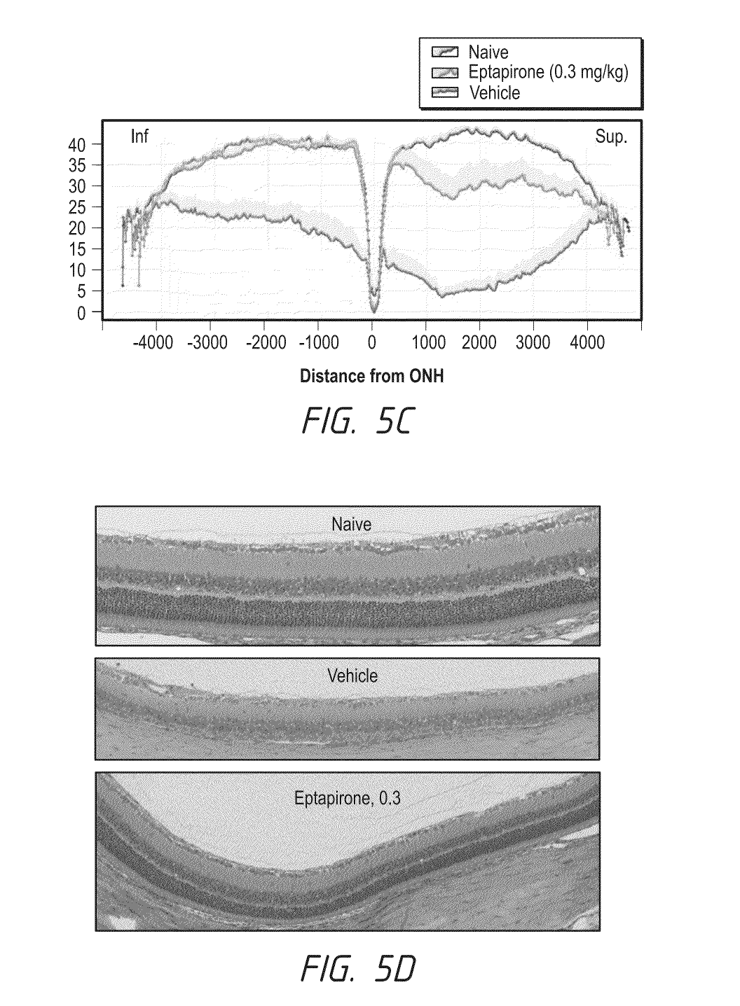

Protection of rod photoreceptors by zalospirone from light induced injury is exemplified in FIG. 4 and by eptapirone in FIG. 5 below. Doses greater of 1 mg/kg provide 100% protection of structural integrity of the outer nuclear layer and 80% of retinal signaling in a blue-light induced damage model in rodent. When the retinal lesion, i.e., loss of photoreceptors is measured in the vertical meridian of the eye it is apparent that maximal damage occurs 1.5 mm superior to the optic nerve head. Treatment with either zalospirone or eptapirone almost entirely prevents this lesion from forming. Images of H&E stained retinas comparing naïve and post-blue light exposed vehicle-, and drug-treated retinae demonstrate that treatment with either compound not only maintains outer nuclear cell count but also normalizes retinal structure in the face of a severe insult.

During the initial stages of geographic atrophy visual acuity may be preserved, but as the atrophic lesion impinges on the central, cone-rich foveal region a rapid and life-altering loss results. In a study by Schatz and McDonald, the rate of significant visual loss (from 20/50 or better to 20/100 or worse) was 8% of eyes per year as atrophic lesions expanded into the fovea from extrafoveal regions. Therefore, protection of cone photoreceptors in addition to rods is a preferred embodiment of this disclosure. While FIG. 4 illustrates the protection of rods by zalospirone, this figure does not specifically exemplify the protection of cones. Protection of cones can be independently measured using specific immunocytochemical and electroretinographic techniques.

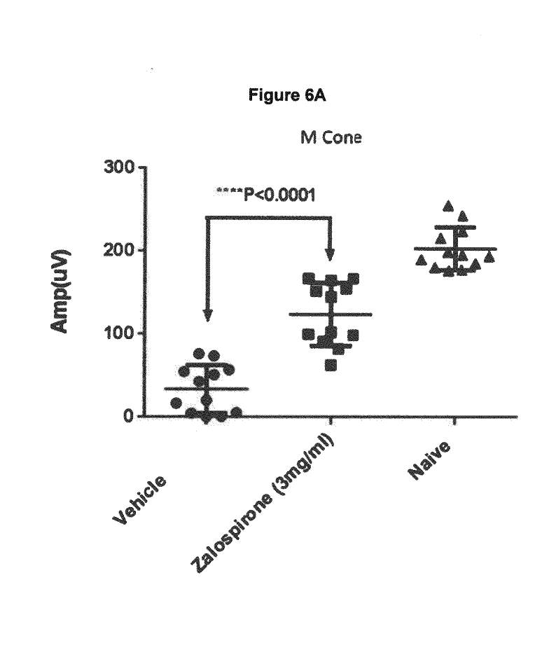

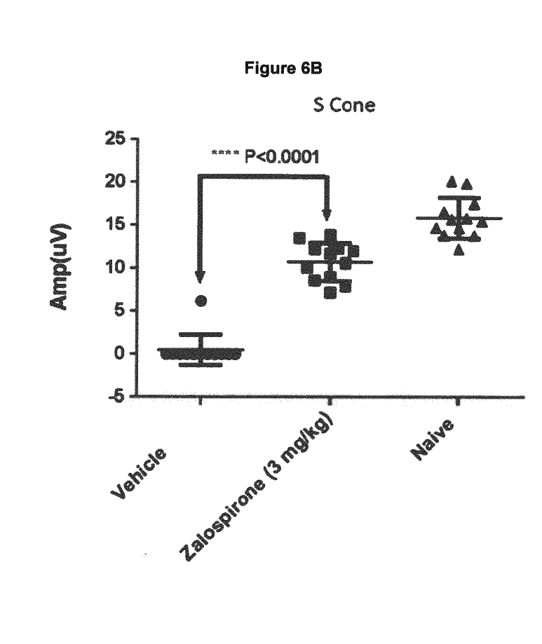

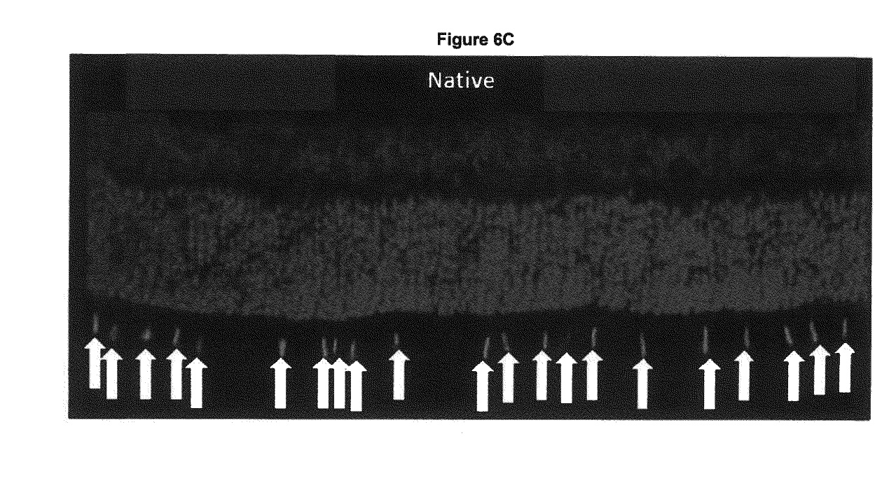

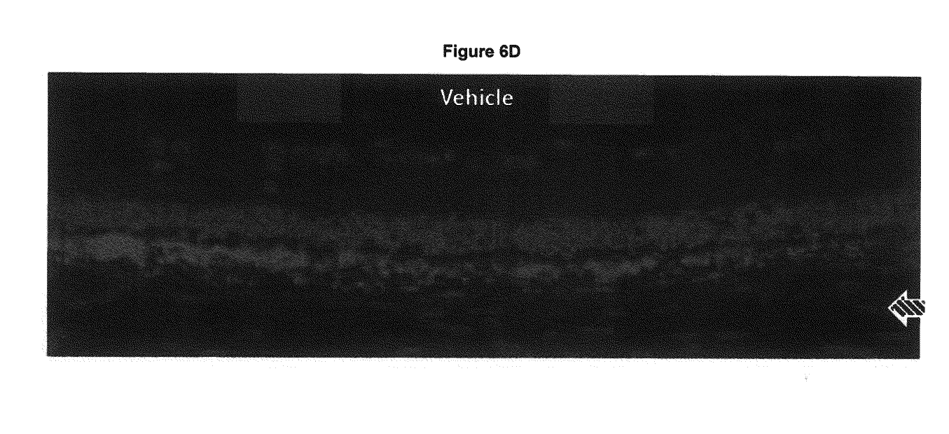

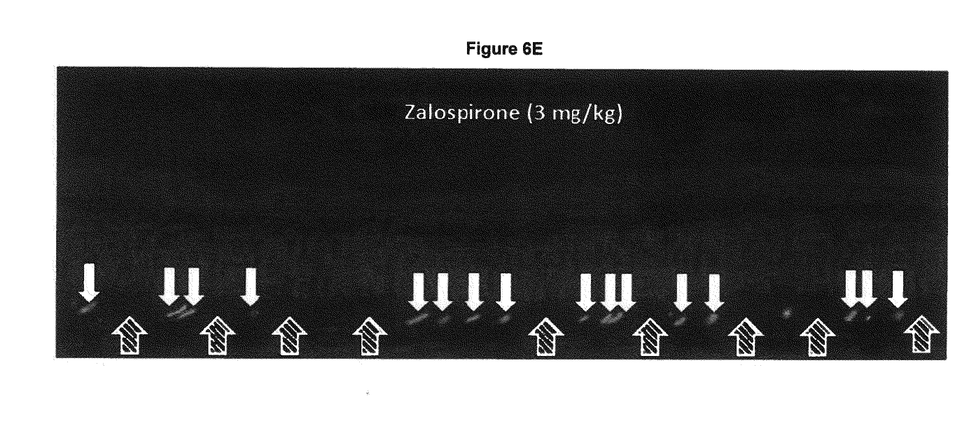

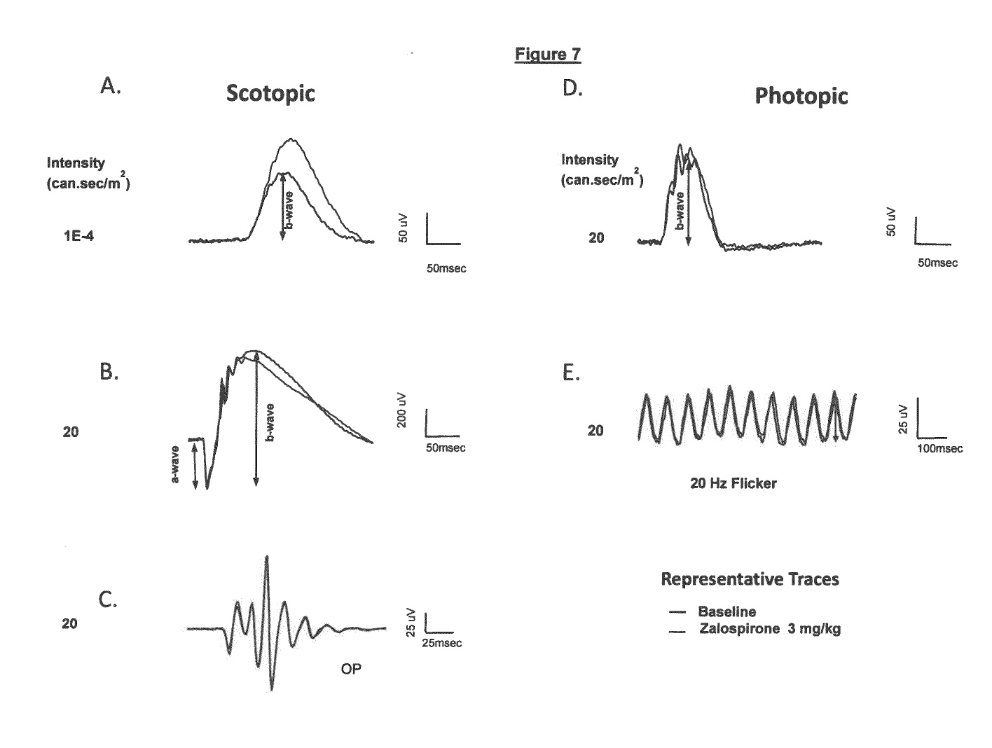

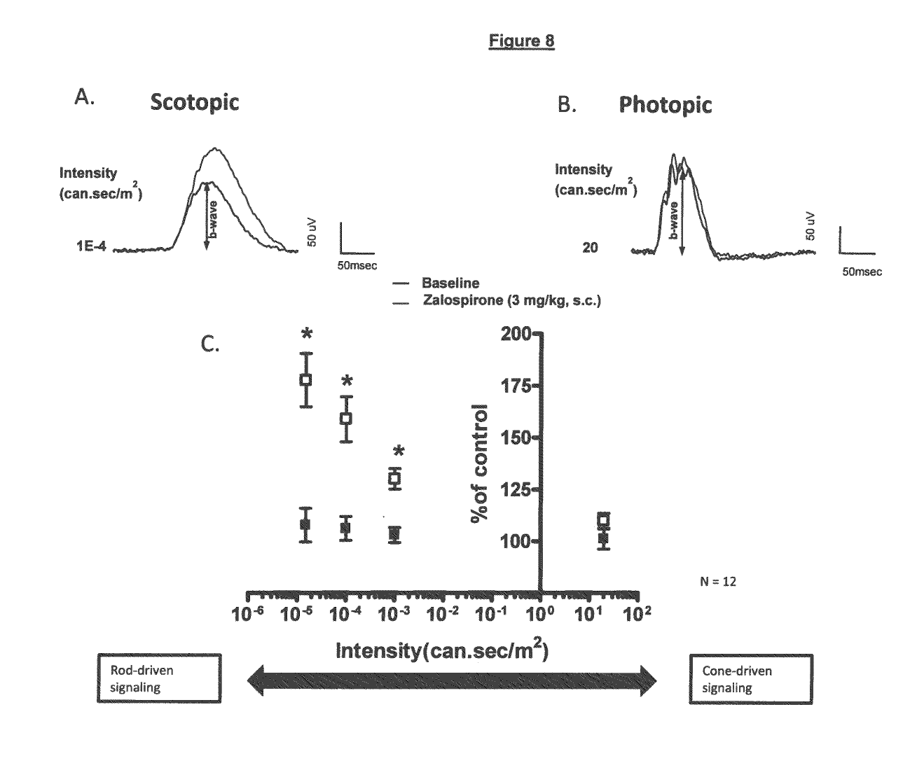

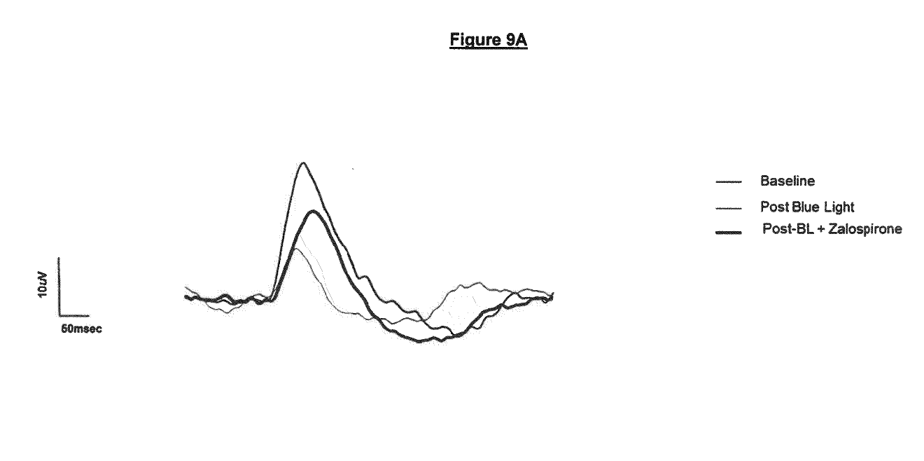

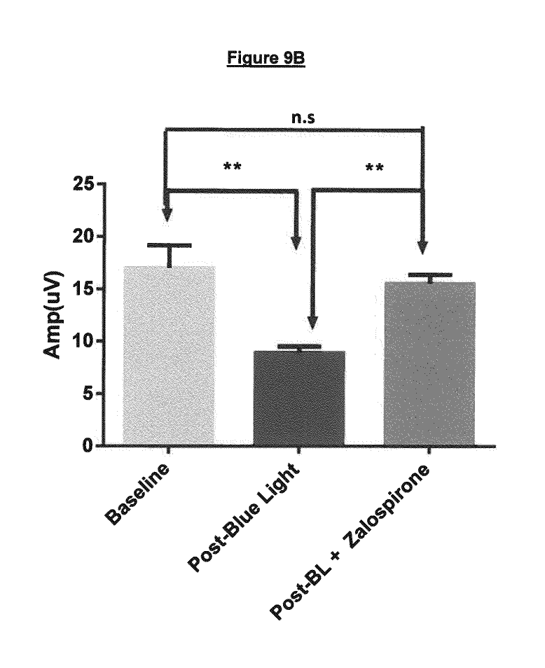

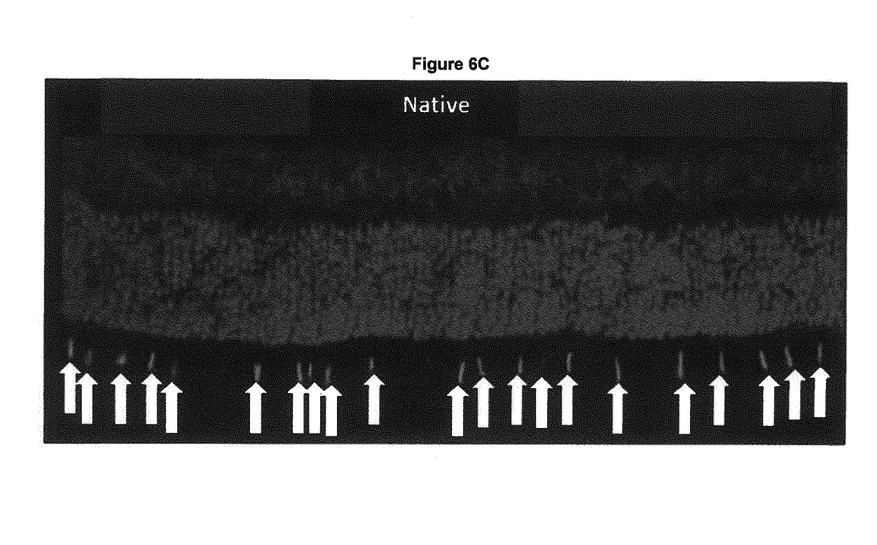

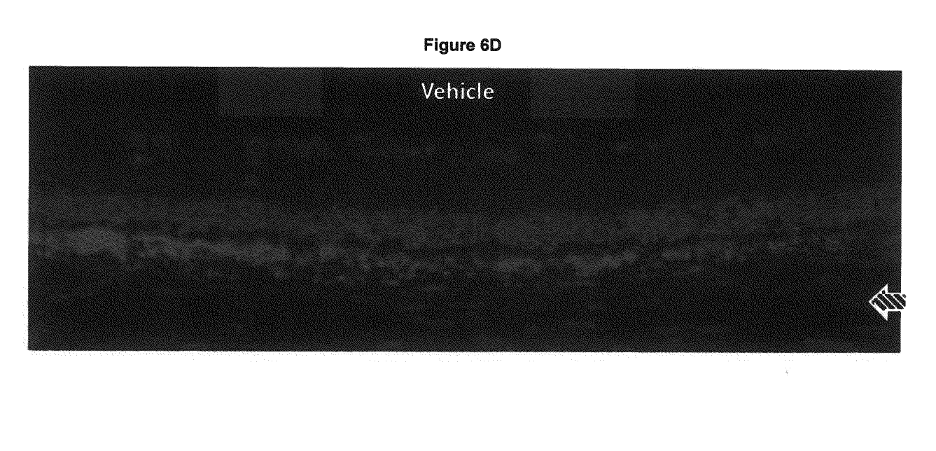

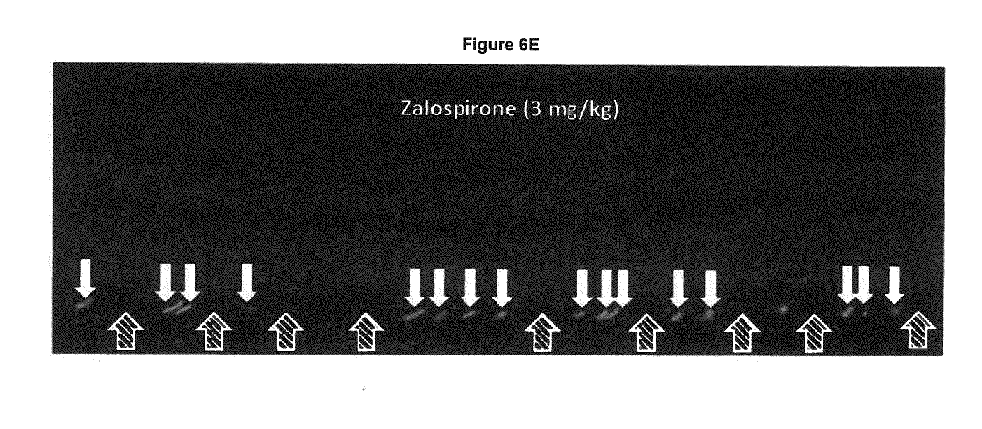

The protection of cone photoreceptors by zalospirone from light induced injury is exemplified in FIG. 6 below. Measurements of the amplitudes of the photopic b-wave using ERG protocols that isolated the activity of each of the two types of cone photoreceptors in the rat show that both are significantly protected from blue light-induced injury by treatment with zalospirone (FIG. 6 A,B). Additionally, labeling cone photoreceptor opsins in transverse retinal sections shows the complete loss of cones in vehicle treated subjects following exposure to blue-light. Whereas, after blue-light exposure in the presence of zalospirone the cone density appears virtually identical to that in naïve animals (FIG. 6 C-E).

In addition to protecting retinal photoreceptors and preserving their function during exposure to an insult, the enhancement or normalization of retinal signaling after injury would be provide significant value in degenerative retinal diseases. The International Society for Clinical Electrophysiology of Vision (ISCEV) provides standards for ascertaining the effects of interventions on retinal signaling. Applying modified ISCEV standards for full-field clinical electroretinography to rat, it was determined that zalospirone selectively enhanced scotopic signaling (FIG. 7). A more detailed analysis of dark adapted animals (FIG. 8) demonstrated that the increase in b-wave amplitude following treatment with zalospirone was produced at stimulus intensities that activate principally rod-driven retinal signaling. Expanding on the observation that scotopic signaling was enhanced in normal animals, it was hypothesized that zalospirone could contribute to the normalization of retinal signaling following injury. FIG. 9 demonstrates that 10 days after a blue light induced-injury, which reduced the amplitude of the scoptopic b-wave by 50%, treatment with zalospirone (3 mg/kg) fully restored the b-wave to its baseline amplitude.

Thus, data based on functional and structural protection of photoreceptors demonstrate that zalospirone and eptapirone is expected to provide protection in rod and cone dystrophies and non-exudative AMD. Furthermore the enhanced rod based signaling demonstrated by zalospirone suggests that these compounds could provide additional benefit in improving vision in patients with impaired vision under conditions of low light.

Throughout this specification reference is made to publications such as US and foreign patent applications, journal articles, book chapters, and others. All such publications are expressly incorporated by reference in their entirety, including supplemental/supporting information sections published with the corresponding references, for all purposes unless otherwise indicated. To the extent that any recitations in the incorporated references conflict with any recitations herein, the recitations herein will control.

The present disclosure is not to be limited in scope by the exemplified embodiments which are intended as illustrations of single aspects of the invention only. Indeed, various modifications and embodiments in addition to those described herein will become apparent to those skilled in the art from the foregoing description. Such modifications are intended to fall within the scope of the appended claims.

What is claimed is:

1. A method of enhancing visual function under low light conditions (scotopic and/or mesopic visual function) in a subject, comprising administering to the subject in need of such enhancement, a therapeutically effective amount of a compound selected from the group consisting of zalospirone and eptapirone; or a pharmaceutically acceptable salt thereof.

2. The method of claim 1, wherein the visual function is selected from the group consisting of visual acuity, visual field, contrast sensitivity, and visual adaptation to different luminance levels.

3. The method of claim 2, wherein the visual function is visual acuity or contrast sensitivity.

4. The method of claim 1, wherein the subject in need of said enhancement of visual function is one who has low/poor visual function resulting from a retinal degeneration or dystrophy.

5. The method of claim 1, wherein the retinal degeneration or dystrophy is selected from the group consisting of age related macular degeneration (AMD) (including wet and dry AMD), geographic atrophy, retinitis pigmentosa, nyctalopia, cone-rod dystrophy, congenital stationary night blindness, macula edema, Stargardts disease cone dystrophy, and, macular edema, retinal detachment and tears, retinal trauma, retinal tumors and retinal diseases associated with said tumors, and diabetic retinopathy.

6. The method of claim 5, wherein the retinal degeneration or dystrophy is selected from the group consisting of age related macular degeneration (AMD) (including wet and dry AMD), geographic atrophy, retinitis pigmentosa, nyctalopia, and cone-rod dystrophy.

7. The method of claim 1, wherein the administration comprises intravitreal administration.

8. A method of protecting photoreceptor cells in a subject, comprising administering to the subject in need of such protection, a therapeutically effective amount of a compound selected from the group consisting of zalospirone and eptapirone; or a pharmaceutically acceptable salt thereof.

9. The method of claim 8, wherein the photoreceptor cells are rods and cones.

10. The method of claim 8, wherein the subject in need of said protection is one who is suffering from a retinal degeneration or dystrophy.

11. The method of claim 8, wherein the retinal degeneration or dystrophy is selected from the group consisting of age related macular degeneration (AMD) (including wet and dry AMD), geographic atrophy, retinitis pigmentosa, nyctalopia, cone-rod dystrophy, macula edema, Stargardt's disease cone dystrophy, macular edema, retinal detachment and tears, retinal trauma, retinal tumors and retinal diseases associated with said tumors, diabetic retinopathy and optic neuropathies.

12. The method of claim 11, wherein the retinal degeneration or dystrophy is selected from the group consisting of age related macular degeneration (AMD) (including wet and dry AMD), geographic atrophy, retinitis pigmentosa, nyctalopia, and cone-rod dystrophy.

13. The method of claim 7, wherein the administration of the compound maintains outer nuclear cell count and/or normalizes retinal structure in the face of a severe insult.

14. The method of claim 8, wherein the administration comprises intravitreal administration.

15. An ocular implant comprising a therapeutically effective amount of a compound selected from the group consisting of zalospirone and eptapirone; or a pharmaceutically acceptable salt thereof.
